(19) United States
(12) Patent Application Publication
(10) Pub. No.: US 2005/0148852 A1
(43) Pub. Date: Jul. 7, 2005

(54) Method for producing result images for an examination object

(76) Inventor: Martin Tank, Heidelberg (DE)

Correspondence Address: HARNESS, DICKEY & PIERCE, P.L.C., P.O.BOX 8910, RESTON, VA, 20195, US

(21) Appl. No.: 11/006,773

(22) Filed: Dec. 8, 2004

(30) Foreign Application Priority Data
Dec. 8, 2003 (DE) 10357205.8

(51) Int. Cl. A61B 5/05

(52) U.S. Cl. 600/407

(57) ABSTRACT

A method is for automatically producing result images for an examination object using section image data. In this case, a target structure is first of all ascertained in the section image data on the basis of a diagnostic questionnaire, and the target structure is taken as a basis for selecting an anatomical norm model whose geometry can be varied using model parameters. The norm model is automatically adapted to the target structure. The section image data are then segmented on the basis of the adapted norm model, with anatomical structures of the examination object which are relevant to the diagnostic questionnaire being separated by selecting all of the pixels within the section image data which are situated within a contour of the adapted norm model and/or at least one model part in line with the relevant structures or have a maximum discrepancy therefrom by a particular value. The relevant structures are then visually displayed separately and/or are stored for later visual display. The document also describes a corresponding image processing system.

The present application hereby claims priority under 35 U.S.C. §119 on German patent application number DE 103 57 205.8 filed Dec. 8, 2003, the entire contents of which are hereby incorporated herein by reference.

### FIELD OF THE INVENTION

The invention generally relates to a method for automatically producing result images for an examination object using section image data from the examination object in question. The invention also generally relates to an image processing system which can be used to carry out such a method.

### BACKGROUND OF THE INVENTION

The result of examinations using modalities which produce section images, such as computer tomographs, magnetic resonance tomographs and ultrasound equipment, normally includes a number of series of section images of the examination object in question. For further planning of the examination and/or in order to produce a diagnosis, these section image data must in many cases be processed further during the examination itself, or immediately after the examination.

The flow of such examinations is normally determined by a diagnostic questionnaire in this case. In most cases, this involves a particular organ or system of organs being examined more closely only after outline images have been prepared.

One example of this is the examination of clinically relevant knee problems in a patient. Following the preparation of relatively few series of section images of the knee, an intermediate diagnosis of any existing pathologies of the internal structures of the knee is first produced and more extensive examinations of the relevant area of the knee are then performed on this basis. Normally, to produce this intermediate diagnosis, a user, for example the radiologist or an MTRA (medical-technical radiological assistant), needs to analyze the individual outline images and then to make a decision about how to proceed further. Producing an intermediate diagnosis of this type requires a time involvement which is not to be underestimated, and this impairs the entire examination workflow.

A further problem is that identifying pathologies of particular internal structures, particularly in the case of very complex anatomical structures, in the section image data can be extremely difficult and requires some experience. It is therefore easy to make incorrect intermediate diagnoses. This may sometimes result in impairment of the quality of the section image examinations.

Various methods are admittedly already known for producing individual models for particular structures of interest in the section images and for using these models to support diagnoses or for intervention planning. Thus, by way of example, WO 99/55233 describes a method for model-based evaluation of ultrasound images of the heart in which an individual model of the heart of the person being examined is produced and evaluated semi-automatically—by adapting a model to three manually detected anatomical landmarks. In addition, DE 103 11 319 A1 describes a method in which an individual 3D model of the heart is produced on the basis of CT images, likewise using three manually Stipulated anatomical landmarks, in order to plan a cardiac intervention procedure.

Furthermore, U.S. 2003/0097219 describes a method in which a model of the left cardiac ventricle is produced semi-automatically on the basis of anatomical landmarks. Finally, WO 00/32106 describes a method for performing a virtual endoscopy using individualized models of the respiratory or digestive tract. However, all of these methods only ever involve the output of just one model, and any diagnosis or intervention planning which is based on this is accordingly highly dependent on the quality of the model produced.

### SUMMARY OF THE INVENTION

It is therefore an object of an embodiment of the present invention to provide an alternative method and an image processing system for automatically producing result images for the examination object using previously produced section image data, which allow diagnoses—particularly intermediate diagnoses for continuing the examination—to be produced significantly more easily, more quickly and more safely.

This object may be achieved by a method and/or by an image processing system.

In line with the inventive method of an embodiment, this involves a target structure of interest first of all being automatically ascertained in the section image data on the basis of a diagnostic questionnaire. This target structure is then taken as a basis for selecting an anatomical norm model whose geometry can be varied using model parameters. In this case, the various anatomical models may be managed in a database, where each organ to be examined has at least one corresponding anatomical norm model which covers this organ.

This norm model is then automatically adapted to the target structure in the section image data, i.e. is individualized on the basis of this target structure. The section image data are then segmented on the basis of the adapted norm model, with relevant anatomical structures of the examination object which are of interest to the diagnostic questionnaire being separated by selecting all of the pixels in the section image data which are situated within a contour of the adapted model and/or at least one model part in line with the relevant anatomical structures or have a maximum discrepancy therefrom by a particular difference value. In this case, the selection may be made such that the pixels in question are removed or that all remaining pixels in the model or model part in question are removed, i.e. the pixels in question are cut out. In this context, “model part” is understood to mean a part of the norm model, for example the base of the skull in a model of the skull. In this case, exactly this model part may correspond to the organ (part) which is actually to be examined. The relevant anatomical structures are then visually displayed separately and/or are stored for later visual display.

In this case, this visual display may be effected in two or three dimensions, for example on the screen of an operating console for the modality in question or for a workstation connected thereto via a network. It is likewise possible to output the result images on a printer, on a filming station or the like. The separate visual display of the relevant anatomical structures may be effected in the form that all of the single parts of the organ in question are shown separately from one another in a result image, for example in the manner of an exploded-view drawing.

In addition, the individual structures may also be shown on individual result images which a person making the diagnosis can view alternately, in succession or in parallel on various printouts, screen windows etc. In the case of a three-dimensional display, this is preferably done such that the user is able to rotate the structures or the individual structure interactively on an appropriate user interface virtually in space and is thus able to view it from all sides. In addition, besides the “SSD” (Surface Shaded Display) presentation type, where simply the surface of the structures is shown, as already mentioned above, it is also possible to use any other presentation types which are respectively most expedient for the individual relevant structures during separate visual display, such as VRT (Volume Rendering Technique), MPR (Multiplanar Reconstruction), MIP (Maximum Intensity Projection) etc.

The proposed method allows the section image data to be segmented on the basis of the norm model, i.e. to be broken down into all of the diagnostically relevant parts. The subsequent separate visual display of the various anatomical structures in the result images makes it an extraordinarily simpler matter to make a correct intermediate diagnosis, particularly for less experienced personnel too. The method therefore results in more rapid production and validation of an intermediate diagnosis during a section image examination, which reduces the overall examination time and at the same time improves the quality of the examination result.

The method may also help to optimize the actual medical diagnosis following the examination. As a departure from the previously known methods described at the outset, this involves visually displaying the actually measured and segmented volume data for the structure of interest and not a model of this structure.

In this context, in contrast to the conventional threshold-value or regional-growth methods such as are described in U.S. Pat. No. 6,556,696 B1, for example, segmentation on the basis of an individualized model has the advantage that this method may also be used in cases in which the structures to be separated cannot be identified by a pronounced sudden change of contrast in the section image data.

To this end, an image processing system based on an embodiment of the invention first requires an interface for receiving the measured section image data, a target-structure ascertainment unit for ascertaining a target structure in the section image data on the basis of a diagnostic questionnaire, a memory device having a number of anatomical norm models, preferably in the form of a database, for various target structures in the section image data, whose geometry may respectively be varied on the basis of particular model parameters, and a selection unit for selecting one of the anatomical norm models in line with the ascertained target structure. In addition, the image processing system requires an adaptation unit for adapting the selected norm model to the target structure in the section image data, a segmentation unit for segmenting the section image data on the basis of the adapted norm model, and in so doing, separating anatomical structures of the examination object which are relevant to the diagnostic questionnaire by selecting all of the pixels within the section image data which are situated within a contour of the adapted norm model or a model part in line with the relevant anatomical structures or have a maximum discrepancy therefrom by a particular difference value.

Finally, a visual display device is required for automatically visually displaying the relevant anatomical structures separately or for storing them in suitable fashion for later visual display. In this context, “visual display device” should be understood to mean a device which conditions the segmented section image data such that the relevant structures are displayed separately from one another and can be viewed individually, for example on a screen or else on other output units connected to the image processing system.

In one preferred variant, while the norm model is being adapted to the target structure a particular discrepancy function is respectively taken as a basis for ascertaining a current discrepancy value between the geometry of the modifying norm model and the target structure. Thus, the adaptation can be performed fully automatically by simply minimizing the discrepancy value.

In this case, the automatic adaptation can take place entirely in the background, which means that the user can address other work and, in particular, can use a console for the image processing system which produces the desired result images to process other image data and/or to control other measurements in parallel. Alternatively, it is possible for the process to be displayed permanently on a screen or a subregion of the screen, for example, during the automatic method, which means that the user can monitor the progress of the adaptation process.

Preferably, the current value of the discrepancy function is displayed to the user. In particular, it is also possible for the discrepancy values to be displayed permanently on the screen, e.g. in a taskbar or the like, while the rest of the user interface is free for other work by the user.

Preferably, the user has the option of intervening in the automatic adaptation process if required and of adjusting individual model parameters manually. In this case, the user is advantageously shown the current discrepancy value, so that when varying the model parameters in question he immediately sees whether and to what extent the geometrical discrepancies are reduced by his actions. In particular, it is also possible in this context to determine individual discrepancy values for each model parameter and to display these instead of an overall discrepancy value or in addition thereto.

A typical example of this is the display of the target structure and/or of the norm model which is to be adapted or at least some of these objects on a graphical user interface on a terminal, with the user being able to use the keyboard or being able to use a pointer device such as a mouse or the like, for example, to adapt a particular model parameter—for example the distance between two points on the model. A progress bar or a similar visually easily recognizable means is then used to show the user the extent to which the discrepancies are reduced by his actions, the display showing, in particular, firstly the total discrepancy of the model and secondly the discrepancies with regard to the adaptation of the specific current model parameter—for example, in the case of a distance between two points in the model, the latter's difference with respect to the distance between the relevant points in the target structures.

In one particularly preferred exemplary embodiment, the segmentation is preceded by an automatic check to determine whether adapting the norm model to the target structure involves a minimum discrepancy value being reached which is below a prescribed threshold value. That is to say that a check is carried out to determine whether the discrepancy between the model and the target structure in the data record is sufficiently small. Only if this is the case is automatic segmentation of the measured data record performed on the basis of the model. Otherwise, the method is aborted for the purpose of further manual processing of the section image data. This reliably prevents excessive discrepancies between the model and the measured data record from causing incorrect automatic segmentation to be performed which could result in incorrect diagnoses on the basis of the automatically segmented and visually displayed anatomical structures.

With very particular preference, it is also possible, besides the simple separate visual display of the relevant anatomical structures, to check these anatomical structures for discrepancies from the norm as well. That is to say that the discrepancies between the anatomical structure in question and an individualized model or model part are ascertained automatically.

Preferably, this is done using a norm model or norm model part which has merely been individualized in a particular manner. When individualizing this comparative norm model, which is to be used for such identification of discrepancies from the norm, it is necessary to ensure that only transformations such that the geometry of the comparative norm model or of the relevant norm model part itself has no pathologies are performed. The discrepancies ascertained can then be visually displayed graphically together with the anatomical structures. By way of example, they may be marked for the user in the visually displayed data record on a screen. In addition, such discrepancies may also be unambiguously displayed to the user by means of an audible signal. It is thus a simple matter for pathologies in the examined anatomical structures to be automatically established and indicated to the user.

In a further development of this method, it is also possible for the examination object to be automatically classified on the basis of the ascertained discrepancies from the norm. By way of example, it can automatically be stipulated whether further examinations are necessary and, if so, what examinations are performed. In this case, it is also an obvious step to present the classification to the user merely as a proposal, so that the user may then agree to the proposal and hence the further examinations are performed without any great complexity, or that the user can simply reject the proposal in order to make an independent decision about whether and what detailed examinations need to be performed, in the conventional manner.

It is fundamentally possible to perform the individualization of the anatomical norm model, i.e. the adaptation to the target structure, using any suitable individualization method. The idea of individualizing an anatomical model may, in general, be formulated in simplified form such that a geometrical transformation is sought—in the case of a three-dimensional model, in line with a three-dimensional transformation—which adapts the model in optimum fashion to an individual computer-tomography, magnetic-resonance-tomography or ultrasound data record. All of the information which can be associated with the geometry of the model is likewise individualized in this case.

During medical image processing, such a method for determining optimum transformation parameters is also referred to as a registration or matching method. In this context, a distinction is normally drawn between what are known as rigid, affinitive, perspective and elastic methods, depending on what geometrical transformation is used. Such registration methods have been used to date, for example, in order to combine two or more images in a common image or in order to adapt anatomical atlases to image data. Various such methods are described, inter alia, in WO 01/45047 A1, DE 693 32 042 T2, WO 01/43070 A1 and DE 199 53 308 A1.

To handle the individualization problem mathematically, a discrepancy function is normally used, as described above, which describes the discrepancy between an arbitrarily transformed model and a section image data record. In this context, the type of discrepancy function is dependent on the respective type of anatomical norm model used.

The digital anatomical norm models which may be used may in principle have a wide variety of designs. One option is, by way of example, to model anatomical structures on a voxel basis, the editing of such volumetric data requiring special software which is normally expensive and not readily available. Another option is modeling using “finite elements”, where a model is normally constructed from tetrahedra. Such models also require special, expensive software, however. What is relatively readily available is simple modeling of anatomical boundary areas using triangulation. The corresponding data structures are supported by many standard programs from the field of computer graphics. Models constructed on the basis of this principle are referred to as “surface-oriented anatomical models”. These are the lowest common denominator for modeling anatomical structures, since appropriate surface models can be derived both from the first-mentioned volumetric models by triangulating the voxels and by converting the tetrahedra from the finite element method into triangles.

It is therefore an obvious step to use surface-oriented models constructed on a triangle basis as norm models. Firstly, this method allows the models to be produced in the simplest and least expensive manner. Secondly, models which have already been produced in another form, particularly the aforementioned volumetric models, can be accepted through appropriate transformation, which means that there is then no need to produce an appropriate model afresh.

To produce such surface models afresh, it is possible, by way of example, to segment section image shots with appropriate complexity using a conventional manual method. The information about the individual structures, for example individual organs, which is obtained in this way may finally be used to generate the models. To obtain human bone models, it is also possible, by way of example, for a human skeleton to be measured using laser scanners or to be scanned and segmented and also triangulated using a computer tomography.

In such models, by way of example, the discrepancy function may be defined on the basis of the method of least squares, this function being used to calculate a measure of the discrepancy from the positions of the transformed model triangles relative to the target structures.

In one particularly preferred exemplary embodiment of the invention, an elastic registration method is used. To find a minimum value for the discrepancy function as quickly as possible, this preferably involves the use of a multistage method. By way of example, in a three-stage method, suitable positioning, i.e. translation, rotation and scaling, may first be used to adapt the model coarsely. Volumetric transformation may then be carried out in a second step in order to achieve better tuning. In a third stage, fine tuning is then performed in order to adapt the model to the structure locally in optimum fashion.

With particular preference, individualization is performed using a hierarchically parameterized norm model in which the model parameters are arranged hierarchically in terms of their influence on the overall anatomical geometry of the model. The norm model is then individualized in a plurality of iteration steps, the number of model parameters which can be set simultaneously in the respective iteration step—and hence the number of degrees of freedom for the model variation—being increased in line with the hierarchical arrangement of the parameters as the number of iteration steps increases.

This method ensures that during the individualization the model parameters which have the greatest influence on the overall anatomical geometry of the model are adjusted first. Only then is it possible to set the subordinate model parameters, which influence only some of the overall geometry, on a gradual basis.

This ensures an effective and consequently time-saving practice during model adaptation, regardless of whether the adaptation is performed fully automatically or whether a user intervenes manually in the adaptation method. In the case of a (partly) manual method, this may be done, by way of example, by virtue of the user being presented, during each iteration step, with the individual model parameters only on the basis of their hierarchical arrangement with respect to the variation, e.g. using a graphical user interface.

Preferably, the model parameters are respectively associated with one hierarchical class. Thus, different model parameters may possibly also be associated with the same hierarchical class since they have approximately the same influence on the overall anatomical geometry of the model. During a particular iteration step, it is then possible to add all of the model parameters in a particular hierarchical class afresh for setting purposes. In a subsequent iteration step, the model parameters in the hierarchical class below that are then added etc.

A model parameter may be associated with a hierarchical class on the basis of a discrepancy in the model geometry which arises when the model parameter in question is altered by a particular value. In this case, in one particularly preferred method, various hierarchical classes have particular ranges of discrepancies, e.g. numerical discrepancy ranges, associated with them. That is to say that, for example to put a parameter into a hierarchical class, this parameter is altered and the resultant discrepancy between the geometrically altered model and the original state is calculated.

In this case, the extent of the discrepancy is dependent on the type of norm model used. The only crucial factor is that an accurately defined extent of discrepancy is ascertained which quantifies as accurately as possible the geometrical alteration on the model before and after the relevant model parameter is varied, in order to ensure a realistic comparison for the influence of the various model parameters on the model geometry. To this end, a uniform step size is used preferably for each parameter type, i.e. for example for range parameters where the distance between two points in the model is varied or for angle parameters where an angle between three points in the model is varied, in order to be able to compare the geometrical influence directly.

The parameters are then simply put into the hierarchical classes by prescribing numerical ranges for this extent of discrepancy. When surface models produced on a triangle basis are being used, the discrepancy between the unaltered norm model and the altered norm model is calculated following variation of a parameter preferably on the basis of the sum of the geometrical distances between corresponding triangles in the models in the various states.

Preferably, a topmost hierarchical class whose model parameters can be set immediately in a first iteration step contains at least the very model parameters whose variation prompts a global alteration to the norm model. These include, by way of example, the total of nine parameters for rotating the entire model around the three model axes, for translation along the three model axes and for scaling the entire model along the three model axes.

The individual model parameters may be hierarchically classified, in principle, during the segmentation of the section image data. In that case, by way of example, each iteration step then first involves a check to determine which further model parameters have the greatest influence on the geometry, and these parameters are then added. Since this has significant associated computation complexity, however, the model parameters are classified or put into the hierarchical order particularly preferably in advance, for example when the norm model is actually produced, but at least before the norm model is stored in a model database or the like for later selection.

That is to say that the model parameters are arranged hierarchically with respect to their influence on the overall anatomical geometry of the model preferably in advance in a separate method for producing norm models, which are then available for use in the cited method for producing result images. In this case, the model parameters may likewise be assigned to corresponding hierarchical classes, a parameter being associated with a hierarchical class again on the basis of a discrepancy in the model geometry which arises when the model parameter in question is altered by a particular value. This removal of the hierarchical arrangement of the model parameters to a separate method for producing a norm model has the advantage that each norm model requires the calculation of the hierarchical order of the model parameters to be performed just once, and hence valuable computation time can be saved during segmentation. The hierarchical order may be stored relatively easily together with the norm model, for example by storing the parameters, arranged in hierarchical classes or combined with appropriate markers or the like, in a file header or at another normalized position in the file, which also contains the further data for the norm model in question.

In one very particularly preferred exemplary embodiment, the model parameters are respectively linked to a position for at least one anatomical landmark for the model such that the model has an anatomically meaningful geometry for each parameter set. Typical examples of this are firstly the global parameters, such as rotation or translation of the overall model, where all of the model parameters have had their position altered to suit one another as appropriate. Examples of other model parameters are the distance between two anatomical landmarks or an angle between three anatomical landmarks, for example for determining a knee position.

Such coupling of the model parameters to anatomical landmarks which have been chosen in medically appropriate fashion has the advantage that there is always the possibility of a diagnostic statement after the individualization. The anatomical specialist literature also gives an exact description of the positions of such anatomical landmarks. Such action therefore simplifies the performance of the segmentation, since a medically trained user, for example a doctor or MTA, is familiar with the anatomical landmarks and these essentially determine the anatomy.

There are various options for automatically ascertaining the target geometry for the object component which is to be separated in the section image data. One alternative is to use the “threshold value method”. This method works by comparing the intensity values (called “Hounsfield values” in computer tomography) of the individual voxels, i.e. of the individual 3D pixels, with a permanently set threshold value. If the value of the voxel is above the threshold value, then this voxel is counted as part of a particular structure.

In the case of magnetic resonance shots, however, this method may be used primarily in contrast agent examinations or to identify the surface of a patient's skin. In the case of computer tomography shots, this method may additionally be used for identifying particular bone structures. This method is not suitable for identifying other tissue structures.

In one preferred method, the target geometry is therefore ascertained at least partly using a contour analysis method. Such contour analysis methods work on the basis of the gradients between adjacent pixels. A wide variety of contour analysis methods are known to a person skilled in the art. The advantage of such contour analysis methods is that the methods can be used in stable fashion both for computer tomography section image data and for magnetic resonance section image data, and for ultrasound section image data.

The target-structure ascertainment unit, the selection unit, the adaptation unit and the segmentation unit and also the visual display unit for the image processing system may be implemented particularly preferably in the form of software on a correspondingly suitable processor in an image computer. This image computer should have an appropriate interface for receiving the image data and a suitable memory device for the anatomical norm models. In this context, this memory device does not necessarily have to be an integrated part of the image computer, rather it is sufficient if the image computer can access a suitable external memory device. For the sake of completeness, it will be mentioned at this juncture that the various components do not absolutely have to be present on one processor or in one image computer, but rather the various components may also be distributed over a plurality of processors or interlinked computers.

Implementing embodiments of the inventive method in the form of software has the advantage that it is also possible to retrofit existing image processing systems relatively easily as appropriate using suitable updates. The inventive image processing system may, in particular, also be an actuation unit for the modality which records the section image data themselves which has the necessary components for processing the section image data on the basis of embodiments of the invention.

### BRIEF DESCRIPTION OF THE DRAWINGS

The invention is explained in more detail below using exemplary embodiments with reference to the appended drawings, in which:

FIG. 1 shows a schematic illustration of an exemplary embodiment of an inventive image processing system which is connected by means of a data bus to a modality and to an image data store,

FIG. 2 shows a flowchart to illustrate one possible sequence of the inventive method,

FIG. 3 shows a flowchart to illustrate a preferred model individualization method in more detail,

FIG. 4 shows an illustration of possible target structures for a human skull in the section image data of a computer tomography,

FIG. 5 shows an illustration of a surface model of a human skull,

FIG. 6a shows an illustration of the target structures shown in FIG. 4 with an as yet unadapted surface norm model as shown in FIG. 5 (with no lower jaw),

FIG. 6b shows an illustration of the target structures and of the norm model shown in FIG. 6a, but with a norm model which has been partly adapted to the target structure,

FIG. 6c shows an illustration of the target structures and of the norm model shown in FIG. 6b, but with a norm model which has been further adapted to the target structure,

FIG. 7a shows an illustration of the skull norm model shown in FIG. 5 which has been visually displayed in a plurality of separate model parts in the form of an exploded-view drawing,

FIG. 7b shows an illustration of part of the skull norm model shown in FIG. 7a from another viewing direction,

FIG. 8 shows an illustration of anatomical markers on a skull norm model as shown in FIG. 5,

FIG. 9 shows an illustration of a surface model of a human pelvis which has been formed on a triangle basis.

### DETAILED DESCRIPTION OF THE EXEMPLARY EMBODIMENTS

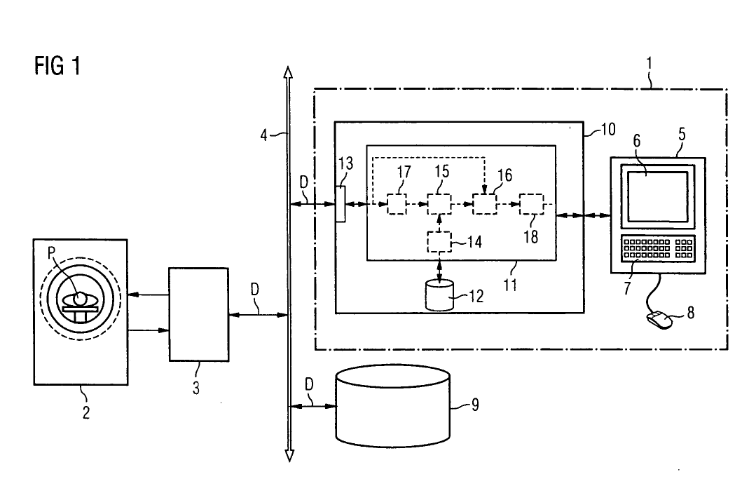

The exemplary embodiment of an inventive image processing system 1 which is shown in FIG. 1 essentially includes an image computer 10 and a console 5, connected thereto, or the like with a screen 6, a keyboard 7 and a pointer device 8, in this case a mouse 8. This console 5 or another user interface may also be used, by way of example, by the user to input the diagnostic questionnaire or to select it from a database containing prescribed diagnostic questionnaires.

The image computer 10 may be a computer of ordinary design, for example a workstation or the like, which may also be used for other image evaluation operations and/or to control image recorders (modalities) such as computer tomographs, magnetic resonance tomographs, ultrasound equipment etc. Fundamental components within this image computer 10 are, inter alia, a processor 11 and an interface 13 for receiving section image data D from a patient P which have been measured by a modality 2, in this case a magnetic resonance tomography 2.

In the exemplary embodiment shown in FIG. 1, the modality 2 is connected to a control device 3 which in turn is connected to a bus 4 to which the image processing system 1 is also connected. In addition, this bus 4 has a mass memory 9 connected to it for buffer-storing or permanently filing the images recorded by the modality 2 and/or the image data D processed further by the image processing system 1. It goes without saying that other components which are present in an ordinary radiological information system (RIS), for example further modalities, mass memories, workstations, output devices such as printers, filming stations or the like, may also be connected to the bus 4 to form a larger network. Similarly, connection to an external network or to further RISs is possible. In this arrangement, all of the data are formatted preferably in the DICOM (DICOM=Digital Imaging and Communication in Medicine) standard for the purpose of communication among the individual components.

The modality 2 is actuated in the usual manner using the control device 3, which also acquires the data from the modality 2. The control device 3 may have a separate console or the like (which is not shown in this case, however) for the purpose of operating it in situ. Alternatively, it is possible for it to be operated via the bus, for example, using a separate workstation which is close by to the modality.

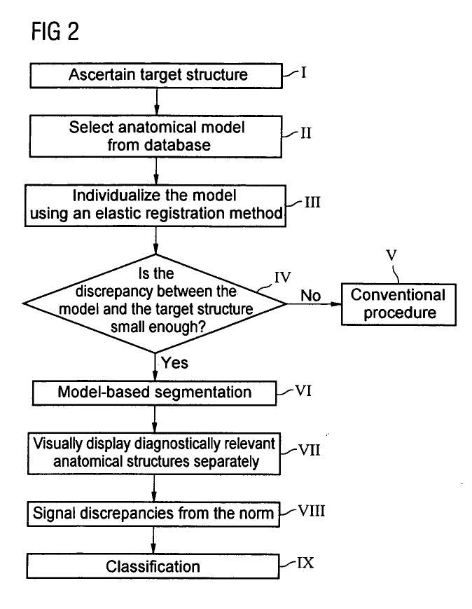

A typical sequence for an inventive method for producing result images of an examination object is shown in FIG. 2.

First, target structures Z within the section image data D are ascertained in a first method step I on the basis of a prescribed diagnostic questionnaire. This is preferably done fully automatically, for example using the aforementioned contour analysis. In the case of certain structures and certain recording methods, it is also possible to use a threshold value method, as already described further above. The section image data D may be supplied, for example directly from the modality 2 or its control device 3, to the image computer 10 via the bus 4. Alternatively, they may be section image data D which have already been recorded some time ago and have been filed in the mass memory 9.

In a step II, a norm model M is then selected in line with the target structure Z. This step may also be performed parallel to or before method step I for ascertaining the target structure, since the type of target structure Z to be ascertained is already known from the diagnostic questionnaire, of course. In this regard, the image computer 10 has a memory 12 containing a wide variety of norm models for different possible anatomical structures. These are normally models which comprise a plurality of model parts.

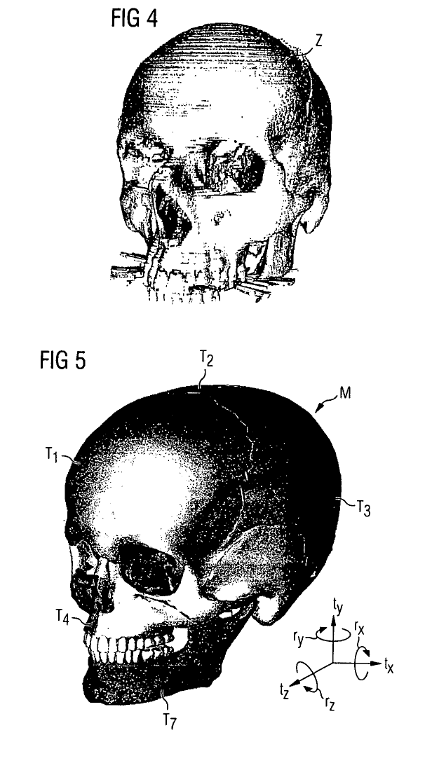

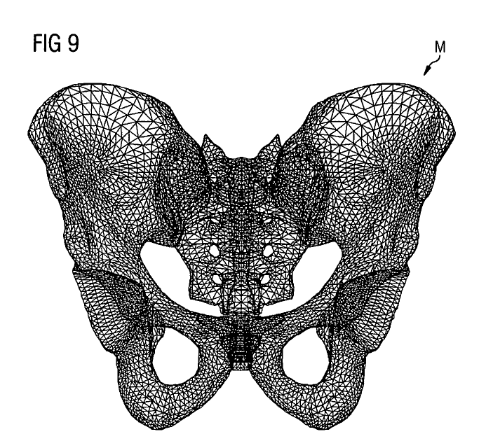

A typical example of this may be explained with reference to a knee examination, where the diagnostic questionnaire is aimed at examining certain structures within the knee. A target structure for the knee is then first ascertained in the recorded section image data, for example the outer bony surface of the knee. An appropriate knee model for this comprises the model parts “femur”, “tibia”, “patella” (kneecap) and the individual meniscuses, for example. By contrast, in the case of a diagnostic questionnaire which relates to the patient's head, for example in order to check the suspicion of skull fracture, the target structure ascertained from the section image data could be the bony surface structure of the skull. Such a target structure which has been obtained from a patient's computer tomography data is shown in FIG. 4. FIG. 5 shows a suitable skull norm model, which includes, inter alia, the frontal bone T₁, the right parietal bone T₂, the left parietal bone T₃, the facial cranium T₄ and the lower jaw T₇. The model is shown with a continuous surface to improve recognizability. In actual fact, the models are constructed on the basis of triangles. A corresponding surface model of a pelvis is shown in FIG. 9.

The appropriate model M is selected using a selection unit 14, and a target structure is ascertained using a target-structure ascertainment unit 17, which in this case are in the form of software on the processor 11 in the image computer 10. This is shown schematically in FIG. 1.

Next, in a method step III, the model is individualized using an “elastic registration method”. Other individualization methods are also possible in principle, however. This adaptation of the norm model M to the target structure Z is performed within an adaptation unit 15 which—as shown schematically in FIG. 1—is likewise in the form of a software module on the processor 11 in the image computer 10.

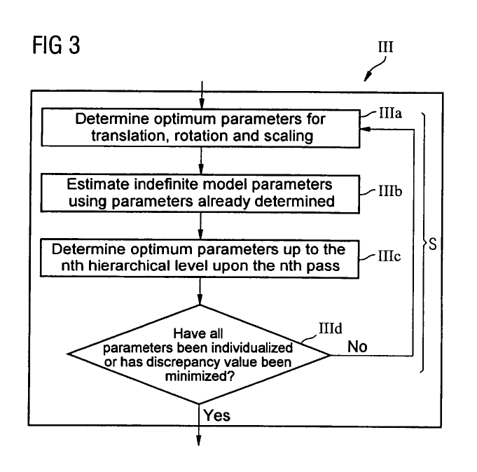

One preferred embodiment of the individualization process is shown schematically in more precise form in FIG. 3 in the form of a flowchart. In this adaptation process, the individual model parameters are varied in a plurality of iteration steps S until ultimately all of the parameters have been individualized or the individualization is sufficient, i.e. the discrepancies between the norm model M and the target structure Z are minimal or are below a prescribed threshold value. In this case, each iteration step S comprises a plurality of process steps IIIa, IIIb, IIIc, IIId, which are performed in the form of a loop.

The loop or the first iteration step S starts at method step IIIa, in which the optimum parameters for translation, rotation and scaling are first determined. These are the parameters in the topmost (subsequently “0th”) hierarchical class, since these parameters affect the overall geometry. The three parameters of the translation tₓ, t_{y}, t_{z} and the three parameters of the rotation rₓ, r_{y}, r_{z} around the three model axes are shown schematically in FIG. 5.

Once this adaptation has gone as far as possible, model parameters which have not yet been set are estimated in a further step IIIb using parameters which have already been determined. That is to say that the settings for superordinate parameters are used to estimate start values for subordinate parameters. One example of this is the estimation of the knee width from the settings for a scaling parameter for the body size. This value is prescribed as an original value for the subsequent setting of the relevant parameter. This allows the method to be speeded up considerably.

The relevant parameters are then set in optimum fashion in method step IIIc.

In the exemplary embodiment shown, the parameters are arranged hierarchically in terms of their influence on the overall anatomical geometry of the model. The greater a parameter's geometric effect, the further up it is in the hierarchy. As the number of iteration steps S increases, the number of model parameters which can be set is increased in line with the hierarchical arrangement in this case.

That is to say that in the first iteration step S or within the first pass of the loop only the parameters of the 1st hierarchical level below the 0th hierarchical level are used to set the model in step IIIc. During the second pass, it is then possible to subject the model to more translation, rotation and scaling again in method step IIIa first. In method step IIIb, the as yet undetermined model parameters in the 2nd hierarchical class are then estimated using already determined parameters which are then added in step IIIc for setting purposes. This method is then repeated n times, with all of the parameters from the nth level being optimized in the nth iteration step, and the last step IIId of the iteration step S in turn settling whether there are still further parameters available which have not been optimized to date.

A new, (n+1)th iteration step then starts in turn, with the model again first being appropriately shifted, rotated or scaled and finally all of the parameters again being able to be set one after the other, in which case the parameters of the (n+1)th class are also available. There is then a fresh check in method step IIId to determine whether all of the parameters have been individualized, i.e. whether there are still parameters which have not yet been optimized, or whether the desired adaptation has already been achieved.

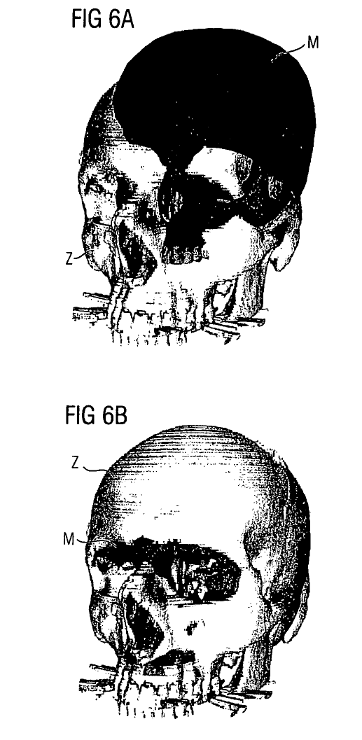

FIGS. 6a to 6c show a very simple case for an adaptation process of this type. This figure shows the model M as a continuous surface again, for the purpose of improved clarity. FIG. 6a shows the target structure Z with the model M moved against it. Simple translation, rotation and scaling gives the image shown in FIG. 6b, in which the model M has already been adapted relatively well to the target structure Z. By setting further, subordinate parameters, the adaptation achieved in FIG. 6c is finally obtained.

The iteration method described above ensures that adaptation takes place in the most time-saving and effective fashion possible. During the adaptation, it is at all times possible to show on the screen 6 of the console 5 both the target structure Z and the associated model M, and also currently calculated discrepancy values or the currently calculated value of a discrepancy function. In addition, the discrepancies may also be visually displayed as shown in FIGS. 6a to 6c. In addition, the discrepancy may also be visually displayed through appropriate coloration.

The subordinate hierarchical classes are obtained from the quantitative analysis of the geometrical influence. To this end, each parameter is altered and the resultant discrepancy in the geometrically altered model from the original state is calculated. This discrepancy may be quantified, by way of example, by the sum of geometrical distances between corresponding model triangles when triangle-based surface models as shown in FIG. 9 are used.

By prescribing numerical ranges for the discrepancy, the parameters can be put into the hierarchical classes. In this case, it is entirely likely that different parameters will fall into the same hierarchical class. This is dependent, inter alia, on the size of the numerical ranges for the discrepancies. As explained above, these parameters in the same hierarchical class are for the first time provided for alteration simultaneously within a particular iteration step S or are automatically altered as appropriate in the case of an automatic adaptation method.

As already mentioned, this method preferably involves the use of model parameters which are connected directly to one or more positions for particular anatomical markers in the model. This firstly has the advantage that only medically appropriate transformations of the model are performed. Secondly, it has the advantage that the medically trained user normally knows these anatomical landmarks and can therefore handle these parameters extremely well.

Examples of such parameters are the positions of the anatomical landmarks L, L₁, L₂ shown on a model of the skull in FIG. 8 or the distances between the individual landmarks, such as the distance d₀ between the anatomical landmarks L₁, L₂ at the center point of the orbital cavities (eye sockets). In order to set this distance d₀ for the orbital cavities during manual intervention in the automatic adaptation process by a user, the user may use a mouse pointer, for example, to select one of the anatomical landmarks L₁, L₂ and to alter its position interactively. The geometry of the model is then automatically shaped as appropriate at the same time.

When varying a model parameter, which covers a distance between two anatomical landmarks in the norm model M, the geometry of the norm model is preferably shaped in a region along a straight line between the anatomical landmarks proportionally to the change of distance. When varying a model parameter which covers an alteration in the position of a first anatomical landmark relative to an adjacent landmark, the geometry of the norm model M is preferably shaped as appropriate at the same time in an area surrounding the relevant first anatomical landmark in the direction of the relevant adjacent landmarks.

In this case, the shaping advantageously decreases as the distance from the relevant first anatomical landmark increases. That is to say that the shaping is greater in the relatively narrow region around the landmark than in the regions which are at a further distance therefrom, in order to achieve the effect shown in the figures. Alternatively, other transformation rules are conceivable, provided that they result in anatomically appropriate transformations. This may be dependent on the respective model selected.

The anatomical markers L, L₁, L₂ on a model of the skull may also be used to illustrate a typical example in which the distances between two landmarks have been put into different hierarchical classes. Hence, the model of the skull shown in FIG. 8 is not only determined by the distance d₀ between the two orbital cavities but it is also parameterized by the distance between the two processi styloidei, which are small bony projections at the base of the skull (not seen in the view in FIG. 8).

In this case, the geometrical effect of the first parameter, which specifies the orbital distance, is greater than the geometrical effect of the second parameter, which indicates the distance between the processi styloidei. This can be examined by virtue of a geometrical alteration of the model for a parameter alteration by one millimeter. Since the processi styloidei are relatively small structures, the geometrical model alteration will be limited to a small region around these bony projections.

This is in contrast to the relatively much larger orbital cavities. When the orbital distance is altered, a multiple component of the model will alter its geometry and will result in an increased discrepancy. For this reason, the parameter of the orbital distance is arranged in a much higher hierarchical class than the alteration of the distance between the processi styloidei, since fundamentally parameters with a greater geometrical range in the parameter hierarchy are higher than parameters with a more local effect.

When all of the settable parameters have finally been individualized or when the discrepancy function has reached its minimum value, method step IV checks whether the individualized norm model's discrepancy from the data record, i.e. from the target structure, is small enough. In this context, it is possible to check, by way of example, whether the discrepancy value which has currently been reached is below a limit value. If this is not the case, the automatic process is terminated and the rest of processing takes place—as shown schematically as method step V in this case—in conventional fashion. That is to say that the image data are then evaluated manually by the user and a manual intermediate diagnosis is produced. Appropriately, in the event of such termination, a corresponding signal is output to the user, which means that the user immediately recognizes that he needs to continue to handle the ongoing process manually.

If, on the other hand, the adaptation of the norm model M to the target structure Z is sufficient, then the segmentation is performed in method step VI. This is done in a separation unit 16 which is likewise—as shown schematically in FIG. 1—in the form of a software module within the processor 11. In this context, all of the pixels within the section image data are selected which are within a contour of the model or a particular model part in line with the anatomical structure which is relevant on the basis of the diagnostic questionnaire. To this end, all other data are erased, for example, which means that only the desired pixels remain.

In method step VII, all of the segmented data are then conditioned fully automatically such that separate visual display of the diagnostically relevant anatomical structures in the form of the desired result images is possible. This is done using a graphical user interface. It is an obvious step to do this using a commercially available program for showing three-dimensional objects, for example by conditioning the data for the separate, relevant (substructures using the visual display unit in line with an interface for such a program.

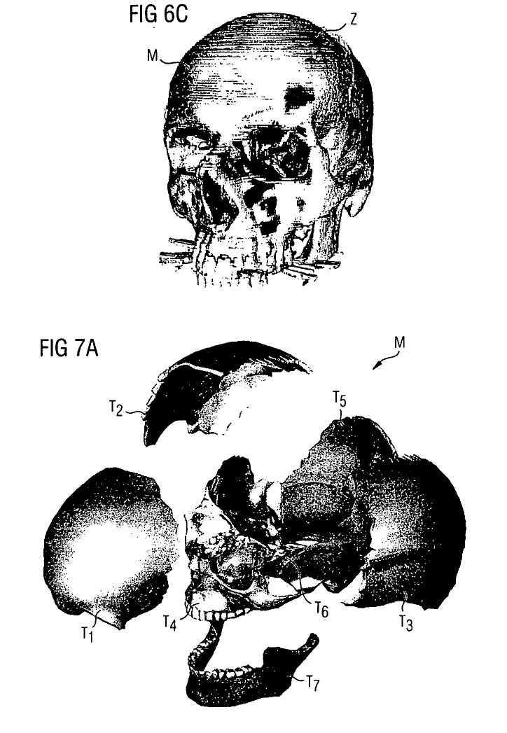

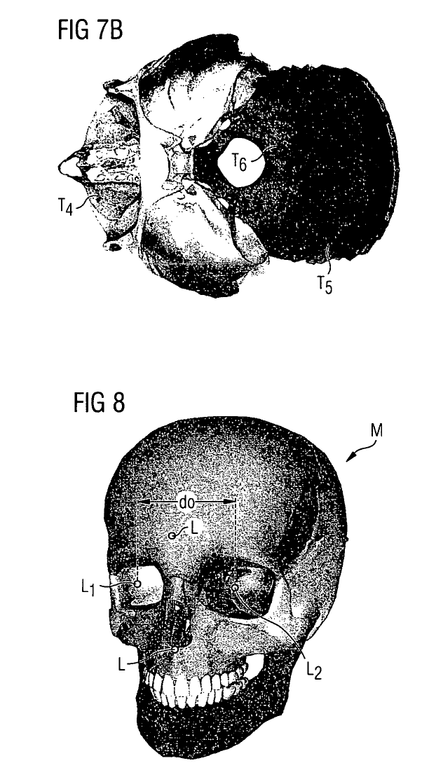

FIGS. 7a and 7b show the form in which—for example when examining the skull—visual display of the relevant structures is possible. Each figure shows the skull norm model shown in FIG. 5. FIG. 7a shows this model M in the manner of an exploded-view drawing, where the fundamental model parts T₁, T₂, T₃, T₄, T₅, T₆, T₇ are shown separately from one another on a result image. These are specifically the frontal bone T₁ (os frontale), the right parietal bone T₂ (os parietale dexter), the left parietal bone T₃ (os parietale sinister), the facial cranium T₄ (viscerocranium), the occipital bone T₅ (os occipitale), the base of the skull T₆ (basis cranii interna), which includes a part of the occipital bone T₅, and the lower jaw T₇ (mandibula). In FIG. 7a, the facial cranium T₄ and the base of the skull T₆ (includes the occipital bone T₅) are still joined to one another as a common part.

All of the substructures or model parts T₁, T₂, T₃, T₄ T₅, T₆, T₇ may be marked separately by the user on a graphical user interface, for example can be “clicked on” using a mouse and viewed separately from all sides by virtually rotating and scaling them in space.

FIG. 7b shows a top view of the cohesive part of the skull, comprising facial cranium T₄ and base of the skull T₆ (includes the occipital bone T₅). As a comparison of the images 7a and 7b with FIG. 5 very quickly shows, the separate visual display of the relevant structures (i.e. including the internal structures) makes it possible to detect pathologies inside a complex structure more easily. Hence, in the illustrated example of a skull examination, even inexperienced medical personnel or even laymen would readily be able to detect a fracture at the base of the skull on a representation as shown in FIG. 7b. By contrast, this is possible only by experienced medical personnel in the case of the classical evaluation of section image data.

In the case of the exemplary embodiment shown in FIG. 2, the visual display is immediate, as in most cases. If the process of execution is running in the background, an audible and/or visual indication is given, for example, that the process has progressed to a stage at which visual display is possible. Alternatively or in addition, the result images produced in this manner, which show the diagnostically relevant anatomical structures separately from one another—or the conditioned data on which these images are based—, can first be buffer-stored, so that they may be retrieved later at any time. The result images may preferably also be output on a printer, a filming station or the like or may be sent via a network to another station in order to be displayed there on a screen or the like.

In the exemplary embodiment shown in FIG. 2, discrepancies from the norm in the various separate structures of a respective associated norm model or model part are also marked in the result images so as to simplify diagnosis by a user. This is preferably done in combination with an audible signal, which signals to the user that there are corresponding discrepancies from the norm at particular locations.

In method step IX, the further examination steps are then stipulated. This may be done automatically on the basis of the established discrepancy from the norm or else manually by the user. In one particularly preferred variant, the discrepancies from the norm are automatically taken as a basis for proposing further examination steps to the user, which the user may either accept or reject or else add to or alter.

The proposed image processing system is therefore used not only for conditioning images for viewing, like normal image processing systems, but also as a model-based expert system which results in faster production and validation of intermediate diagnoses in the course of section image examinations. The inventive method and image processing system may therefore assist in significantly reducing the overall examination time and also in improving the quality of the examination results. In particular, the actual medical diagnosis following an examination may also be optimized using the outlined approach, since the identification of possible pathologies is made much simpler for the doctor as a result of the provision of result images with separate relevant anatomical structures—possibly together with previously provided markings for discrepancies from the norm.

At this juncture, it will once again be expressly pointed out that the system architectures and processes illustrated in the figures are merely exemplary embodiments which may readily be altered in detail by a person skilled in the art. In particular, the control device 3 (provided that it is equipped with an appropriate console, for example) may also have all corresponding components of the image computer 10 so that the image processing based on the inventive method can be performed there directly. In this case, the control device 3 itself therefore forms the inventive image processing system, and a further workstation or a separate image computer is not necessary.

It is otherwise an obvious step to retrofit a process control unit based on the invention to existing image processing systems in which known post-processing processes have already been implemented, so that these installations may also be used in line with the inventive method described above. In many cases, it may also be sufficient to update the control software with suitable control software modules.

Exemplary embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A method for automatically producing result images for an examination object using section image data from the examination object, comprising:
ascertaining a target structure in the section image data on the basis of a diagnostic questionnaire;
using the target structure as a basis for selecting an anatomical norm model with geometry variable using model parameters;
automatically adapting the norm model to the target structure in the section image data;
segmenting the section image data on the basis of the adapted norm model, with anatomical structures of the examination object which are relevant to the diagnostic questionnaire being separated by selecting all of the pixels within the section image data which are either situated within a contour of at least one of the adapted norm model and at least one model part in line with the relevant anatomical structures, or have a maximum discrepancy therefrom by a particular difference value; and
at least one of visually displaying the relevant anatomical structures separately and storing the relevant anatomical structures for later visual display.

2. The method as claimed in claim 1, wherein during the adaptation, a particular discrepancy function is respectively taken as a basis for ascertaining a current discrepancy value between the modified norm model and the target structure.

3. The method as claimed in claim 2, wherein the model parameters are altered in an automatic adaptation method such that the discrepancy value is minimized.

4. The method as claimed in claim 2, wherein the segmentation is preceded by an automatic check to determine whether adapting the norm model to the target structure involves a minimum discrepancy value being reached which is below a prescribed threshold value and the method otherwise being aborted for the purpose of further manual processing of the section image data.

5. The method as claimed in claim 1, wherein at least one separate anatomical structure of the examination object is automatically checked for discrepancies from the norm.

6. The method as claimed in claim 5, wherein ascertained discrepancies from the norm are at least one of visually displayed graphically and signaled to a user audibly with the associated separate anatomical structure.

7. The method as claimed in claim 5, wherein the examination object is automatically classified on the basis of ascertained discrepancies from the norm.

8. The method as claimed in claim 1, wherein the norm model is adapted in a plurality of iteration steps to the target structure in the section image data using model parameters which are in a hierarchical order in terms of their influence on the overall anatomical geometry of the model, and wherein the number of settable model parameters is increased in line with their hierarchical order as the number of iteration steps increases.

9. The method as claimed in claim 8, wherein the model parameters are respectively associated with one hierarchical class.

10. The method as claimed in claim 9, wherein a model parameter is associated with a hierarchical class on the basis of a discrepancy in the model geometry which arises when the model parameter in question is altered by a particular value.

11. The method as claimed in claim 10, wherein various hierarchical classes include particular value ranges of discrepancies associated with them.

12. The method as claimed in claim 1, wherein the norm models used are surface models generated on a triangular basis.

13. The method as claimed in claim 1, wherein the model parameters are respectively linked to a position for at least one anatomical landmark such that the model has an anatomically meaningful geometry for each parameter set.

14. The method as claimed in claim 1, wherein the target structure in the section image data is ascertained at least partly automatically using a contour analysis method.

15. A computer program product which can be loaded directly into a memory in a programmable image processing system, having program codes, in order to perform all of the steps of a method as claimed in claim 1 when the program product is executed on the image processing system.

16. An image processing system for automatically producing result images for an examination object using section image data from the examination object, comprising:
an interface for receiving the measured section image data;
a target-structure ascertainment unit for ascertaining a target structure in the section image data on the basis of a diagnostic questionnaire;
a memory device having a number of anatomical norm models for various target structures in the section image data, whose geometry may respectively be varied on the basis of particular model parameters;
a selection unit for selecting one of the anatomical norm models in line with the ascertained target structure;
an adaptation unit for adapting the selected norm model to the target structure in the section image data;
a segmentation unit for segmenting the section image data on the basis of the adapted norm model and, in so doing, separating anatomical structures of the examination object which are relevant to the diagnostic questionnaire by selecting all of the pixels within the section image data which at least one of are situated within a contour of the adapted norm model or a model part in line with the relevant anatomical structures and have a maximum discrepancy therefrom by a particular difference value; and
a visual display unit for at least one of automatically visually displaying the relevant anatomical structures separately and storing the relevant anatomical structures for later visual display.

17. A modality for measuring section image data for an examination object, comprising an image processing system as claimed in claim 16.

18. The method as claimed in claim 3, wherein the segmentation is preceded by an automatic check to determine whether adapting the norm model to the target structure involves a minimum discrepancy value being reached which is below a prescribed threshold value and the method otherwise being aborted for the purpose of further manual processing of the section image data.

19. The method as claimed in claim 6, wherein the examination object is automatically classified on the basis of ascertained discrepancies from the norm.

20. The method as claimed in claim 2, wherein at least one separate anatomical structure of the examination object is automatically checked for discrepancies from the norm.

21. The method as claimed in claim 20, wherein ascertained discrepancies from the norm are at least one of visually displayed graphically and signaled to a user audibly with the associated separate anatomical structure.

22. The method as claimed in claim 20, wherein the examination object is automatically classified on the basis of ascertained discrepancies from the norm.

23. A computer program product which can be loaded directly into a memory in a programmable image processing system, having program codes, in order to perform all of the steps of a method as claimed in claim 2 when the program product is executed on the image processing system.
